(19) United States
(12) Patent Application Publication
(10) Pub. No.: US 2002/0147158 A1
(43) Pub. Date: Oct. 10, 2002

(54) Composition and method for preventing and treating sinusoidal obstruction syndrome and radiation-induced liver disease

(75) Inventor: Laurie DeLeve, Playa del Rey, CA (US)

Correspondence Address: Jennifer M. Phelps, McCutchen, Doyle, Brown & Enersen, LLP, 18th Floor, Three Embarcadero Center, San Francisco, CA, 94111, US

(73) Assignee: University of Southern California, Los Angeles, CA

(21) Appl. No.: 10/086,072

(22) Filed: Feb. 27, 2002

Related U.S. Application Data
(60) Provisional application No. 60/271,780, filed on Feb. 27, 2001.

(51) Int. Cl. A61K 31/704; A61K 31/195

(52) U.S. Cl. 514/34; 514/562

(57) ABSTRACT

The method for using matrix metalloproteinase (“MMP”) inhibitors to prevent and treat Sinusoidal Obstruction Syndrome (“SOS”). In particular, the present invention provides a method of preventing and treating chemotherapy- and radiation-induced liver disease. This invention can be given prophylatically to patients who are receiving high dose chemotherapy and/or radiation and who are at risk for SOS or radiation-induced liver disease. This method may also be used to treat patients therapeutically who have developed SOS or radiation-induced liver disease. Because the development of chemotherapy or radiation-induced liver disease limits patient eligibility for several chemotherapeutic drugs, the present invention increases patient eligibility for many of these drugs.

### CROSS-REFERENCE TO RELATED APPLICATIONS

This application claims the benefit of U.S. Provisional Application No. 60/271,780, filed Feb. 27, 2001, the entire disclosure of which is hereby incorporated by reference in its entirety for all purposes.

### STATEMENT REGARDING FEDERALLY SPONSORED RESEARCH

This invention was made with government support under NIDDK, Grant DK46357 awarded by the National Institutes of Health.

### FIELD OF THE INVENTION

This invention relates to the use of matrix metalloproteinase (MMP) inhibitors in the prevention and treatment of Sinusoidal Obstruction Syndrome; in particular the present invention relates to the prevention and treatment of chemotherapy- and radiation-induced liver complications.

### BACKGROUND OF THE INVENTION

The present invention is directed to the use of matrix metalloproteinase (“MMP”) inhibitors to prevent and treat Sinusoidal Obstruction Syndrome (“SOS”). SOS, also known as hepatic venoocclusive disease, was first diagnosed in cases of liver disease caused by the ingestion of herbal teas or food sources containing pyrrolizidine alkaloids from Crotalaria, Heliotropium and Senecio or from the consumption of bread made from inadequately winnowed wheat contaminated by seeds from these plants. With the modern development of chemotherapy, cases of SOS developed from the long-term use of azathioprine for immunosuppression after renal and liver transplantation and from the use of chemotherapeutic agents. Liver complications of chemotherapy are seen most commonly after high dose chemotherapy, with or without total body irradiation, or high dose radiation to the liver. Liver toxicity is not an uncommon side effect of high-dose chemotherapy. Liver toxicity also occurs after chemotherapy and/or liver irradiation when there is no bone marrow transplantation and hence, conditioning regimens used for marrow ablation are the most common cause of SOS.

SOS is a common complication of chemotherapy with gemtuzumab ozogamicin² or actinomycin D,³ or after long-term immunosuppression with azathioprine in kidney or liver transplantation patients. Other chemotherapeutic agents associated with liver toxicity and SOS include dacarbazine, cytosine arabinoside, mithramycin, 6-thioguanine, urethane, indicine N-oxide, alone and in combination. Milder forms of liver disease from chemotherapy which share the key aspect of sinusoidal endothelial cell injury include nodular regenerative hyperplasia, sinusoidal dilatation and peliosis hepatis. Combinations of irradiation and chemotherapy have also led to the development of SOS. For example, treating nephroblastoma (Wilms' tumor) with dactinomycin and abdominal irradiation has led to SOS.

Radiation-induced liver disease is a condition that shares some of the features of SOS, although there are differences in clinical presentation, histology and time course. Radiation-induced liver disease is seen with radiation doses in excess of 30 to 35 Gy in adults.

SOS has significant morbidity and mortality. The severity of SOS can be classified as mild (SOS is clinically obvious, but requires no treatment and resolves completely), moderate (SOS that causes signs and symptoms requiring treatment such as diuretics or pain medications, but resolves completely) or severe (SOS that requires treatment but that does not resolve before death or day 100.^{15, 16, 17, 22} Some patients have subclinical liver damage, evinced by histologic evidence of liver toxicity in the absence of clinical signs and symptoms.¹⁸ Despite deep jaundice, patients with severe SOS seldom die of liver failure, but rather from renal and cardiopulmonary failure.^{15, 16, 23, 24}

A clinically useful model for predicting the outcome of SOS after cyclophosphamide-based regimes is derived from rates of increase of both bilirubin and weight in the first two weeks following transplantation.²⁵ Furthermore, a poor prognosis correlates with higher serum AST and ALT values, higher wedged hepatic venous pressure gradient, development of portal vein thrombosis, doubling of the baseline serum creatinine, and decreasing oxygen saturation.^{19, 20, 21, 14, 26} There is currently no prophylactic treatment for either SOS or radiation-induced liver disease, and there are no proven therapeutic remedies with high efficacy. The only therapeutic modality with some proven efficacy is the combination of heparin plus tissue plasminogen activator. However, this combination can only be safely used in a very limited group of patients and has efficacy in less than 30% of this limited population of patients.

SOS is the dose-limiting toxicity for several chemotherapeutic drugs and limits patient eligibility. A prophylactic treatment of SOS would have a significant impact on the ability to use high dose chemotherapy. Development of therapies to treat SOS after onset of the disease would be of value in unexpected cases of chemotherapy-induced liver disease.

The molecular events have been best characterized in the rat monocrotaline model. Monocrotaline, the pyrrolizidine alkaloid found in Crotalaria, is one of the best-studied toxins involving SOS.^{5, 6, 4} The monocrotaline model of SOS has the same histologic characteristics as the human disease, as well as the same “clinical features,” i.e., hyperbilirubinemia, hepatomegaly, and ascites formation. In this model, the first morphologic change noted by electron microscopy is loss of the sinusoidal endothelial cell fenestration and the appearance of gaps in the sinusoidal endothelial cell barrier.⁸ Studies with in vivo microscopy and confirmation by electron microscopy have shown that sinusoidal endothelial cells round up, and red blood cells begin to penetrate into the space of Disse beneath the rounded up endothelial cells and dissect off the sinusoidal lining. The sloughed sinusoidal lining cells (i.e., Kupffer cells, sinusoidal endothelial cells, and stellate cells) embolize downstream and obstruct sinusoidal flow.⁷ By the time hepatocyte necrosis is observed, there is extensive denudation of the sinusoidal lining. Early on, there is loss of Kupffer cells, but there is a significant influx of monocytes within the sinusoids, which exacerbates the obstruction of sinusoidal flow by the embolized sinusoidal lining cells. The rounding up or swelling of sinusoidal endothelial cells is the initiating event in SOS and leads to dissection of the sinusoidal lining, which embolizes and blocks the microcirculation.

The initial change to the sinusoidal endothelial cell is morphologically similar to the change in these cells in cold preservation injury. In studying cold preservation cells, Strasberg et al. showed that the upregulation of matrix metalloproteinases (“MMPs”) is involved in changes to the sinusoidal cell.¹¹ Prior to the present invention, it was unknown in the art whether MMPs may be involved in SOS. The inventors of the present invention discovered the relationship between MMPs and the development of SOS. With the discovery of the mechanism that initiates SOS, the inventors were able to develop therapies for the prevention and treatment of SOS and radiation-induced liver disease.

### SUMMARY OF THE INVENTION

The present invention relates in general to the use of MMP inhibitors in the prevention and treatment of liver disease. Accordingly, the present invention provides means to prevent and treat SOS and radiation-induced liver disease.

In a first aspect of the invention, a method is provided for preventing and treating SOS.

In another aspect of the invention, a method is provided for preventing and treating liver complications of chemotherapy, including SOS, nodular regenerative hyperplasia, peliosis hepatis, immunosuppression-induced hepatic venoocclusive disease, and sinusoidal dilatation. It is also an objective of this invention to provide a means to prophylactically treat radiation-induced liver disease.

It is another object of this invention to provide a means to increase patient eligibility for multiple chemotherapeutic drugs by preventing SOS.

### BRIEF DESCRIPTION OF THE DRAWING

FIG. 1. Prevention of SOS by MMP Inhibition

This FIGURE describes the effect of MMP inhibition in the in monocrotaline-induced model of SOS. Injury is rated as absent (−) or as one, two or three plus. The overall score reflects central vein (CV) endothelial damage, hemorrhage and coagulative necrosis: 2-3 points is considered mild SOS, 4-6 points is considered moderate SOS and 7-9 points is severe disease. The MMP2/MMP9 inhibitor used is 2-[(4-biphenylylsulfonyl)amino]-3-phenyl-propionic acid.

As can be seen, on day four, monocrotaline induces severe SOS. This is completely prevented by 2-[(4-biphenylylsulfonyl)amino]-3-phenyl-propionic acid and by doxycycline. On the other hand, the two chemically modified tetracyclines, anhydrotetracycline and isochlorotetracycline, which are doxycycline analogues that are weak MMP inhibitors, do not prevent SOS.

### DETAILED DESCRIPTION OF THE INVENTION

All scientific terms are to be given their ordinary meanings as understood by those of skill in the art, unless an alternate meaning is set forth below. In case of conflict, the definitions set forth in this specification shall control.

In the present invention, the term “Sinusoidal Obstruction Syndrome” or “SOS” is synonymous with the term “hepatic venoocclusive disease.”

In the present invention, it is disclosed that an explanation for the rounding up of sinusoidal endothelial cells may be due to increased activity of MMPs. Because MMPs degrade extracellular matrix, increased MMP activity on the ablumenal side of the sinusoidal endothelial cell would allow the cells to let loose from the space of Disse. In the experimental model, de novo synthesis of MMP-9 (gelatinase B) and increased MMP-9 activity occur 12 hours after monocrotaline, which coincides with rounding up of the sinusoidal endothelial cells.¹² Inhibition of MMP activity completely prevents SOS. MMP expression and activity are regulated by redox status and can be suppressed by glutathione and N-acetylcysteine.²⁷⁻³⁰ Thus, the protective effect of glutathione and N-acetylcysteine appears to be due to inhibition of MMP activity.

The present invention extrapolates from the above model to disclose a method for using matrix metalloproteinase (MMP) inhibitors to prevent or treat SOS and radiation-induced liver disease. The present invention discloses that doxycycline, an MMP inhibitor, completely prevented SOS in a rat model and with human subjects. Further experimentation showed that this was a class effect as the MMP-2 MMP-9 inhibitor, 2-[(4-biphenylsulfonyl)amino]-3-phenyl-propionic acid (BPP) also completely inhibits SOS. A number of other MMP inhibitors will also be effective in the prevention and treatment of SOS and radiation-induced liver disease. For example, Marimastat, Prinomastat and RS-130,830 are potent inhibitors of the MMPs that are increased in the monocrotaline model of SOS. CGS 27023A, Solimastat, BAY 12-9566, Ro 32-3555, BMS-272591, Ilomastat, D2163 are also MMP inhibitors that could be used in humans. Metastat, Neovastat, and Periostat also have potential therapeutic uses in treating and preventing SOS and radiation-induced liver disease.

As described in EXAMPLE I below, the protective effect of both doxycline and of 2-[(4-biphenylsulfonyl)amino]-3-phenyl-propionic acid is dose-dependent, further supporting the biologic mechanism of action. In our animal model the gelatinolytic activity was greatly increased early on in hepatic venoocclusive disease and this increase in gelatinolytic activity could be attributed to MMP9.¹²

The inventors of the present invention have demonstrated that in the rat monocrotaline model, there is actin depolymerization in sinusoidal endothelial cells and that this in turn leads to increased MMP activity.¹³ The causality of the actin depolymerization and increased MMP activity is confirmed by the demonstration that prevention of F-actin depolymerization prevents the monocrotaline-induced increase in matrix metalloproteinase activity. In vitro studies with the various populations of liver cells in vitro have also confirmed that the matrix metalloproteinase activity originates in the sinusoidal endothelial cell rather than in hepatocytes, Kupffer cells or stellate cells.

The following examples are intended to illustrate but not limit the present invention. The methods of the present invention can be further modified for uses such as the identification of drug and diagnostic therapies.

### EXAMPLE I

The present invention provides methods for using matrix metalloproteinase inhibitors to prevent and treat chemotherapy-induced liver disease, such as SOS and radiation-induced liver disease. These studies were done in an in vivo model of monocrotaline-induced hepatic venoocclusive disease which closely resembles the human disease.⁸ Two commercially available MMP inhibitors were tested in the in vivo rat model of hepatic venoocclusive disease: doxycycline and the MMP-2/MMP-9 inhibitor, 2-[(4-biphenylsulfonyl)amino]-3-phenyl-propionic acid. Doxycycline, 15 mg/kg was given twice daily by gavage prior to onset of the disease and continued until the time of sacrifice. 2-[(4-biphenylsulfonyl)amino]-3-phenyl-propionic acid, 200 μg/hour was infused into the portal circulation until the time of sacrifice. A systematic scoring system was devised to review all the changes associated with hepatic venoocclusive disease.

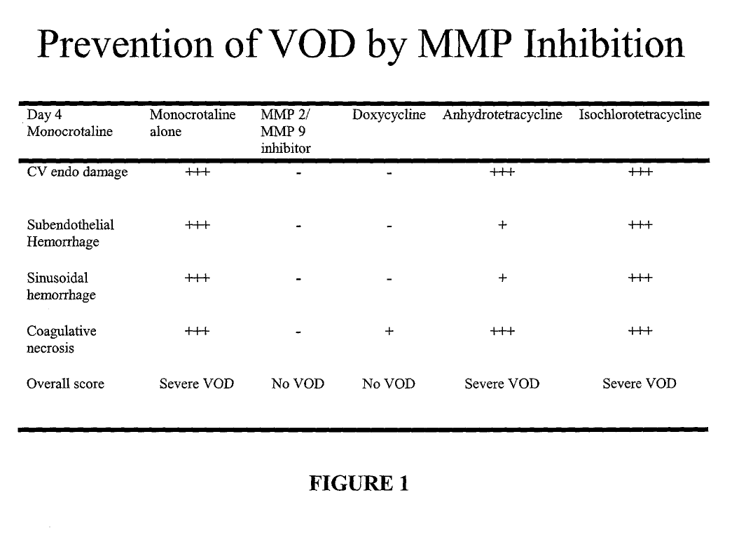

The rats that were treated with the MMP inhibitors were sacrificed on day 4, which is the time-point with most severe disease in this model of hepatic venoocclusive disease when no therapeutic interventions are used. All the livers from the rats treated with doxycycline or with 2-[(4-biphenylsulfonyl)amino]-3-phenyl-propionic acid were examined blindly by a pathologist according to the scoring system and the pathologist was able to confirm an absence of hepatic venoocclusive disease with doxycycline 15 mg/kg twice daily by gavage or with 2-[(4-biphenylsulfonyl)amino]-3-phenyl-propionic acid, 200 μg/hour infused intraportally by osmotic minipump (see FIG. 1). Both doxycycline and 2-[(4-biphenylsulfonyl)amino]-3-phenyl-propionic acid were administered at various doses: lower doses than those mentioned above provided partial protection, whereas the doses listed above completely prevented liver changes in the in vivo model of hepatic venoocclusive disease. Both of these MMP inhibitors prevent hepatic venoocclusive disease in a dose-dependent manner, these showing that this is a class effect of MMP inhibitors and characteristic of MMP inhibition.

In further support of the effect of MMP inhibitors, two analogues of doxycycline were tested for their ability to prevent SOS, anhydrotetracycline and isochlorotetracycline. These two compounds are chemically similar to doxycycline but have little inhibitory effect on MMPs. These analogues had minimal protective effect in the in vivo model (see FIG. 1). Furthermore, in the animal model for hepatic venoocclusive disease, the gelatinolytic activity in liver tissue is greatly increased early on and this increase in gelatinolytic activity could be attributed to MMP9. MMP9 mRNA and MMP9 proenzyme also increase very early in the course of disease. No increased gelatinolytic activity could be found in the hepatic vein effluent indicating it is not a non-specific activity in the circulation. In this disease, as MMP9 activity increases, sinusoidal endothelial cell are rounded up resulting in the loss of sinusoidal integrity, which compromises liver microcirculation. Inhibition of the initial rounding up of the sinusoidal endothelial cell by inhibition of matrix metalloproteinases prevents the whole cascade of events.

It will be understood by those skilled in the art that the foregoing illustrates the presently preferred embodiments of the present invention and that modifications may be made in order to accomplish specific ends which do not depart from the spirit of the present invention which is to be limited only by the following claims.

### REFERENCES

1. DeLeve L D, Shulman H M, McDonald G B. Toxic injury to hepatic sinusoids: sinusoidal obstruction syndrome (venoocclusive disease). Sem.Liver Dis. 2002;22(1):623-38.

2. Rajvanshi P, Shulman H M, Sievers E L, McDonald G B. Hepatic sinusoidal obstruction following Gemtuzumab Ozogamicin (Mylotarg®). Blood 2002:in press.

3. DeLeve L D. Liver Function and Hepatotoxicity in Cancer. In: Holland J F, Frei E, Bast R C, Jr., Kufe D W, Pollock R E, Weichselbaum R R, editors. Cancer Medicine. 5th ed. Hamilton, Ontario, Canada: B.C. Decker Inc; 2000. p. Chapter 151.

4. DeLeve L D. Dacarbazine toxicity in murine liver cells: a novel model of hepatic endothelial injury and glutathione defense. J.Pharmacol.Exp.Ther. 1994;268:1261-70.

5. DeLeve L D, Wang X, Kuhlenkamp J F, Kaplowitz N. Toxicity of azathioprine and monocrotaline in murine sinusoidal endothelial cells and hepatocytes: the role of glutathione and relevance to hepatic venooclusive disease. Hepatology 1996;23:589-99.

6. DeLeve L D. Cellular target of cyclophosphamide toxicity in the murine liver: role of glutathione and site of metabolic activation. Hepatology 1996;24:830-7.

7. DeLeve L D, Ito Y, Machen N W, McCuskey M K, Wang X, McCuskey R S. Embolization by sinusoidal lining cells causes the congestion of hepatic venoocclusive disease. Gastroenterol. 2000;118:A2345.

8. DeLeve L D, McCuskey R S, Wang X, Hu L, McCuskey M K, Epstein R B, et al. Characterization of a Reproducible Rat Model of Hepatic Veno-occlusive Disease. Hepatology 1999;29:1779-91.

9. Wang X, Kanel G C, DeLeve L D. Support of sinusoidal endothelial cell glutathione prevents hepatic veno-occlusive disease in the rat. Hepatology 2000;31:428-34.

10. DeLeve L D, Ito Y, Machen N W, McCuskey M K, McCuskey R S. Sinusoidal dissection and embolization blocks the hepatic microcirculation in hepatic venoocclusive disease. In: Hepatology; 1999; 1999. p. 574A.

11. Upadhya A G, Harvey R P, Howard T K, Lowell J A, Shenoy S, Strasberg S M. Evidence of a role for matrix metalloproteinases in cold preservation injury of the liver in humans and in the rat. Hepatology 1997;26:922-8.

12. DeLeve L D, Wang X, Tsai J, Kanel G, Tokes Z. Prevention of Hepatic Venooclusive Disease in the rat by inhibition of matrix metalloproteinases. Gastroenterol. 2001;120:A54.

13. Lamé M W, Jones A D, Wilson D W, Dunston S K, Segall H J. Protein targets of monocrotaline pyrrole in pulmonary artery endothelial cells. J.Biol.Chem. 2000;275(37):29091-9.

14. Read, A E, Weisner R H, LaBrecquie D R, et al. Hepatic venocclusive disease associated with renal transplantation and azathiprine therapy. Ann Intern Med 1986;104:651-655.

15. McDonald G B, Hinds M S, Fisher L B, et al. Venocclusive disease of the liver and multiorgan failure after bone marrow transplantation: a cohort study of 355 pateints. Ann Intern Med. 1993;118:255-267.

16. Jones R J, Lee K S, Berschorner W E, eta 1. Veno-occlusive disease of the liver following bone marrow transplantation. Transplantation 1987;44:778-783.

17. Carreras E, Bertz H, Arcese W, et al. Incidence and outcome of hepatic venoocclusive disease after blood or marrow transplantation: a prospective cohort study of the European group for blood and marrow transplantation. Blood 1998;92:3599-3604.

18. Shulman H M, McDonald G B, Matthews D, et al. An analysis of hepatic venocclusive disease and centrilobular hepatic degeneration following bone marrow transplantation. Gastroenterology 1980;79:1178-1191.

19. Strasser S I, McDonald S J, Schoch H G, et al. Severe hepatocellular injury after hematopoietic cell transplant: incidence and etiology in 2136 consecutive patients [Abstract]. Hepatology 2000;32:299.

20. Kikuchi K, Rudolph R, McDonald G B. Portal vein thrombosis after heatopoietic cell transplant: incidence, treatment, and outcome. Hepatology 2000; 32:405 (Abs).

21. Carreras E. Granena A, Navasa M, et al. Transjugular liver biopsy in bone marrow transplantation. Bone Marrow Transplant 1993;11:21-26.

22. McDonald G B, Sharma P, Matthews D E, et al. The clinical course of 53 patients with venocclusive disease of the liver after marrow transplantation. Transplantation 1985;36:603-608.

23. Wingard J R, Mellits E D, Jones R J, et al. Association of hepatic veno-occlusive disease with interstitial pneumonitis in bone marrow transplant receipients. Bone Marrow Transplant 1989;4:685-689.

24. Zager R A, O'Quigley J, Zager B K, et al. Acute renal failure following bone marrow transplantation; A retrospective study of 272 patients. Am J Kidney Dis 1989;13:210-216.

25. Bearman S I, Anderson G L, Mori M, et al. Venoocclusive disease of the liber: development of a model for predicting fatal outcome after marrow transplantation. J Clin Oncol 1993;11:1729-1736.

26. Bearman S I, Lee J L, Baron A E, et al. Treatment of hepatic venocclusive disease with recombinant human tissue plasminogen activator and heparin in 42 marrow transplant patients. Blood 1997;89:1501-1506.

27. Cai T, Fassina G, Morini M, et al. N-acetycysteine inhibits endothelial cell invasion and angiogenesis. Lab Invest 1999;79:1151-1159.

28. Tyagi S C, Ratajska A, Weber K T. Myocardial matrix metalloproteinase(s): localization and activation. Mol Cell Biochem 1993;126;49-59.

29. Tyagi S C, Kumar S, Borders S. Reduction-oxidation (redox) state regulation of extracellular matrix metalloproteinases and tissue inhibitors in cardiac normal and transformed fibroblast cells. J Cell Biochem 1996;61:139-151.

30. Upadya G A, Strasberg S M. Glutathione, lactobionate, and histidine: cryptic inhibitors of matrix metalloproteinases contained in University of Wisconinsin and histidine/tryptophan/ketoglutarate liver preservation solutions. Hepatology 200;31:1115-1122.

We claim:

1. A method for preventing or treating Sinusoidal Obstruction Syndrome (“SOS”) comprising administering a matrix metalloproteinase (“MMP”) inhibitor.

2. A method for preventing or treating chemotherapy- or radiation-induced liver disease comprising administering a matrix metalloproteinase (“MMP”) inhibitor.

3. The method of claim 2, wherein said chemotherapy-induced liver disease includes SOS, nodular regenerative hyperplasia, peliosis hepatis, immunosuppression-induced hepatic venoocclusive disease, and sinusoidal dilatation.

4. The method of claim 1 or 2, wherein said MMP inhibitor is doyxcycline or 2-[(4-biphenylsulfonyl)amino]-3-phenyl-propionic acid.

5. The method of claim 4, wherein said MMP inhibitor is doyxcycline.

6. The method of claim 5 wherein 15 mg/kg of said doyxcycline is administered twice daily.

7. The method of claim 4, wherein said MMP inhibitor is 2-[(4-biphenylsulfonyl)amino]-3-phenyl-propionic acid.

8. The method of claim 7 wherein 100-200 mg/hour of said 2-[(4-biphenylsulfonyl)amino]-3-phenyl-propionic acid is administered.

9. The method of claim 1 or 2 wherein said MMP inhibitor is administered for up to 4 weeks.

10. The method of claim 1 or 2, wherein said MMP inhibitor is Marimastat, Prinomastat, RS-130,830, CGS 27023A, Solimastat, BAY 12-9566, Ro 32-3555, BMS-272591, Ilomastat, D2163, Metastat, Neovastat, or Periostat.

11. A method for preventing or treating chemotherapy or radiation induced liver disease comprising administering an effective dose of a matrix metalloproteinase (“MMP”) inhibitor selected from doyxcycline or 2-[(4-biphenylsulfonyl)amino]-3-phenyl-propionic acid.

12. The method of claim 11, wherein said MMP inhibitor is doyxcycline.

13. The method of claim 12, wherein 15 mg/kg of said doyxcycline is administered twice daily.

14. The method of claim 11, wherein said MMP inhibitor is 2-[(4-biphenylsulfonyl)amino]-3-phenyl-propionic acid.

15. The method of claim 14, wherein 100-200 mg/hour of said 2-[(4-biphenylsulfonyl)amino]-3-phenyl-propionic acid is administered.

16. A method for preventing or treating chemotherapy or radiation induced liver disease comprising administering 15 mg/kg of doyxcycline twice daily.

17. A method for preventing or treating chemotherapy or radiation induced liver disease comprising administering 100-200 mg/hour of 2-[(4-biphenylsulfonyl)amino]-3-phenyl-propionic acid.
